(19) United States
(12) Patent Application Publication
(10) Pub. No.: US 2019/0159708 A1
(43) Pub. Date: May 30, 2019

(54) ANALYTE INDICATOR INTEGRATED WITH A CATALYTICALLY ACTIVE MATERIAL

(71) Applicant: Senseonics, Incorporated, Germantown, MD (US)

(72) Inventors: Mark Mortellaro, Germantown, MD (US); Philip Huffstetler, Germantown, MD (US); Tina HyunJung Kim, Germantown, MD (US); Sanat Mohanty, Germantown, MD (US)

(73) Assignee: Senseonics, Incorporated, Germantown, MD (US)

(21) Appl. No.: 16/202,751

(22) Filed: Nov. 28, 2018

Related U.S. Application Data
(60) Provisional application No. 62/591,255, filed on Nov. 28, 2017.

(51) Int. Cl. A61B 5/1486; A61B 5/1459; A61B 5/07

(52) U.S. Cl. CPC A61B 5/14865; A61B 5/1459; A61B 5/14532; A61B 2562/162; A61B 5/076

(57) ABSTRACT

An analyte sensor may include a sensor housing and an analyte indicator element embedded within and/or covering at least a portion of the sensor housing. The analyte indicator element may include a porous base having an interior surface and an exterior surface. The analyte indicator may include a catalytically active material disposed on at least one of the interior and exterior surfaces of the porous base, in which the catalytically active material catalyzes the degradation of reactive oxygen species. The analyte indicator may include a polymer unit polymerized onto or out of the porous base and an analyte sensing element attached to the polymer unit or copolymerized with the polymer unit.

### CROSS-REFERENCE TO RELATED APPLICATION

The present application claims the benefit of priority to U.S. Provisional Application Ser. No. 62/591,255, filed on Nov. 28, 2017, which is incorporated herein by reference in its entirety.

### BACKGROUND

### Field of Invention

The present invention relates generally to sensors for implantation or insertion within a living animal and measurement of an analyte in a medium within the living animal. Specifically, the present invention relates to sensors having a catalytically active material incorporated in an analyte indicator.

### Discussion of the Background

A sensor may include an analyte indicator, such as, for example, indicator molecules embedded or polymerized in or onto a polymer graft (i.e., layer or matrix). If a sensor is implanted in the body of a living animal, the animal's immune system begins to attack the sensor. For instance, if a sensor is implanted in a human, white blood cells attack the sensor as a foreign body, and, in the initial immune system onslaught, neutrophils are the primary white blood cells attacking the sensor. Macrophages and giant cells may further attack the sensor. The defense mechanism of neutrophils and other white blood cells includes the release of highly oxidative substances known as reactive oxygen species (ROS), such as hydrogen peroxide (H₂O₂), hydroxyl radical (OH^{.}), hypochlorite (OCl⁻), peroxynitrite (OONO⁻), and superoxide (O₂⁻).

ROS, such as hydrogen peroxide, may degrade indicator molecules. For instance, in indicator molecules having a boronate group, hydrogen peroxide may degrade the indicator molecules by oxidizing the boronate group, thus disabling the ability of the indicator molecule to bind glucose.

There is presently a need in the art for improvements in reducing analyte indicator degradation.

### SUMMARY

The present invention overcomes the disadvantages of prior systems by providing, among other advantages, reduced analyte indicator degradation caused by exposure to ROS while allowing the analyte indicator to retain its chemical, optical, and physical properties in the presence of compression.

One aspect of the invention may provide an analyte indicator. The analyte indicator may include a porous base having an interior surface and an exterior surface. The analyte indicator may include a catalytically active material disposed on at least one of the interior and exterior surfaces. The catalytically active material may catalyze the degradation of reactive oxygen species (ROS). The analyte indicator may include a polymer unit polymerized onto or out of the porous base. The analyte indicator may include an analyte sensing element attached to the polymer unit or copolymerized with the polymer unit.

In some embodiments, the porous base may include one or more of nylon, cellulose, cellulose acetate, polypropylene, polyethylene, poly(ethylene terephthalate), poly(ether sulfone), poly(vinylidene difluoride), and poly(tetrafluoroethylene). In some embodiments, the polymer unit may include a polyethylene glycol (PEG) unit. In some embodiments, the catalytically active material may include one or more of platinum, iridium, palladium, manganese oxide, thiol and/or disulfide containing compounds, and catalase. In some embodiments, the analyte sensing element may include one or more indicator molecules configured to reversibly bind to an analyte and exhibit one or more detectable properties indicative of whether analyte is bound. In some embodiments, the porous base may be flexible.

In some embodiments, the analyte indicator may retain its chemical, optical, and physical properties in the presence of compression. In some embodiments, the analyte sensing element may include one or more indicator polymer chains, and the one or more indicator polymer chains may include one or more indicator molecules configured to reversibly bind to an analyte and exhibit one or more detectable properties indicative of whether analyte is bound. In some embodiments, the analyte indicator may further include a coating of catalytically active material on the analyte indicator. In some embodiments, the coating of catalytically active material on the analyte indicator may be sputtered on the analyte indicator.

In some embodiments, the analyte indicator may further include a scavenging material disposed on at least one of the interior and exterior surfaces of the porous base. In some embodiments, the scavenging material may be configured to consume ROS. In some embodiments, the scavenging material may include one or more of the following: boronic acid containing compounds, di-acid containing compounds, tocopherol and its derivatives, and ascorbic acid and its derivatives.

One aspect of the invention may provide a sensor. The sensor may include a sensor housing and an analyte indicator element. The analyte indicator element may be embedded within and/or covering at least a portion of the sensor housing. The analyte indicator may include a porous base having an interior surface and an exterior surface. The analyte indicator may include a catalytically active material disposed on at least one of the interior and exterior surfaces. The catalytically active material may catalyze the degradation of reactive oxygen species (ROS). The analyte indicator may include a polymer unit polymerized onto or out of the porous base. The analyte indicator may include an analyte sensing element attached to the polymer unit or copolymerized with the polymer unit.

In some embodiments, the sensor may include a light source configured to emit excitation light to the indicator element and a photodetector configured to receive fluorescent light emitted by the indicator element. In some embodiments, the sensor may include a coating of catalytically active material on the analyte indicator. In some embodiments, the coating of catalytically active material on the analyte indicator may be sputtered onto the analyte indicator.

In some embodiments, the sensor may further include a scavenging material disposed on at least one of the interior and exterior surfaces of the porous base. In some embodiments, the scavenging material may be configured to consume ROS. In some embodiments, the scavenging material may include one or more of the following: boronic acid containing compounds, di-acid containing compounds, tocopherol and its derivatives, and ascorbic acid and its derivatives.

One aspect of the invention may provide an analyte indicator. The analyte indicator may include a porous base. The analyte indicator may include an indicator polymer chain attached or polymerized onto or out of the porous base. The analyte indicator may include one or more indicator molecules attached to the indicator polymer chain. The analyte indicator may include a catalytically active material disposed on at least one of the interior and exterior surfaces of the porous base. The catalytically active material may be configured to catalyze the degradation of ROS.

In some embodiments, the indicator polymer chain may be a first indicator polymer chain, and the analyte indicator may further include a second indicator polymer chain attached or polymerized onto or out of the porous base and indicator molecules attached to the second indicator polymer chain. In some embodiments, the porous base may comprise one or more of nylon, cellulose, cellulose acetate, polypropylene, polyethylene, poly(ethylene terephthalate), poly(ether sulfone), poly(vinylidene difluoride), and poly(tetrafluoroethylene). In some embodiments, the catalytically active material may comprise one or more of platinum, iridium, palladium, manganese oxide, thiol and/or disulfide containing compounds, and catalase. In some embodiments, the analyte sensing element may include one or more indicator molecules configured to reversibly bind to an analyte and exhibit one or more detectable properties indicative of whether analyte is bound.

In some embodiments, the porous base may be flexible. In some embodiments, the analyte indicator may retain its chemical, optical, and physical properties in the presence of compression. In some embodiments, the analyte indicator may further include a coating of catalytically active material on the analyte indicator. In some embodiments, the coating of catalytically active material on the analyte indicator may be sputtered on the analyte indicator.

In some embodiments, the analyte indicator may further include a scavenging material disposed on at least one of the interior and exterior surfaces of the porous base. In some embodiments, the scavenging material may be configured to consume ROS. In some embodiments, the scavenging material may include one or more of the following: boronic acid containing compounds, di-acid containing compounds, tocopherol and its derivatives, and ascorbic acid and its derivatives.

One aspect of the invention may provide a sensor including a sensor housing and an analyte indicator embedded within and/or covering at least a portion of the sensor housing. The analyte indicator may include a porous base. The analyte indicator may include an indicator polymer chain attached or polymerized onto or out of the porous base. The analyte indicator molecule may include one or more indicator molecules attached to the indicator polymer chain. The analyte indicator may include a catalytically active material disposed on at least one of the interior and exterior surfaces of the porous base. The catalytically active material may be configured to catalyze the degradation of ROS.

In some embodiments, the sensor may include a coating of catalytically active material on the analyte indicator. In some embodiments, the sensor may further include a scavenging material disposed on at least one of the interior and exterior surfaces of the porous base. In some embodiments, the scavenging material may be configured to consume ROS. In some embodiments, the scavenging material may include one or more of the following: boronic acid containing compounds, di-acid containing compounds, tocopherol and its derivatives, and ascorbic acid and its derivatives.

Another aspect of the invention may provide a sensor including a sensor housing, an analyte indicator, and a coating of catalytically active material. The analyte indicator may be embedded within and/or covering at least a portion of the sensor housing. The coating of catalytically active material may be on an exterior surface of the analyte indicator. The coating of catalytically active material may be configured to catalyze the degradation of ROS. The analyte indicator may include a porous base, one or more indicator molecules, and a catalytically active material. The one or more indicator molecules may be configured to exhibit one or more detectable properties based on an amount or concentration of an analyte in proximity to the indicator molecules. The catalytically active material may be disposed on at least one of the interior and exterior surfaces of the porous base. The catalytically active material may be configured to catalyze the degradation of ROS.

In some embodiments, the sensor may further include a scavenging material disposed on at least one of the interior and exterior surfaces of the porous base. In some embodiments, the scavenging material may be configured to consume ROS. In some embodiments, the scavenging material may include one or more of the following: boronic acid containing compounds, di-acid containing compounds, tocopherol and its derivatives, and ascorbic acid and its derivatives.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various embodiments of the subject matter of this disclosure. In the drawings, like reference numbers indicate identical or functionally similar elements.

FIG. 1 is a schematic view of a sensor system, which includes an implantable sensor and a sensor reader, embodying aspects of the present invention.

FIG. 2 illustrates a perspective view of a sensor embodying aspects of the present invention.

FIG. 3 illustrates an exploded view of a sensor embodying aspects of the present invention.

FIGS. 4 and 5 illustrate perspective views of sensor components within the sensor body/shell/capsule of a sensor embodying aspects of the present invention.

FIG. 6 illustrates a side view of a sensor embodying aspects of the present invention.

FIG. 7 illustrates a cross-sectional end view of a sensor embodying aspects of the present invention.

FIG. 8 is a macroscale interpretation of an analyte indicator embodying aspects of the present invention, in which polymer units are attached to the porous base and then modified to contain either an indicator molecule or one or more indicator polymer chains yielding a branched system.

FIG. 9 is a blown-up view of a polymer unit containing indicator polymer chains embodying aspects of the present invention.

FIG. 10 illustrates an analyte indicator embodying aspects of the present invention, in which one or more indicator polymer chains are attached or polymerized onto or out of a base membrane layer.

FIG. 11 shows the base monomer make-up of a grafted linear copolymer in which R1, R2, and R3 are hydrophilic acrylate-based monomers such as but not limited to 2-hydroxyethyl methacrylate (HEMA), poly(ethylene glycol) methacrylate (PEGMA), and/or acrylic/methacrylic acid embodying aspects of the present invention.

FIG. 12 is a graph showing the results of in vitro oxidative stability testing of analyte indicators embodying aspects of the present invention.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

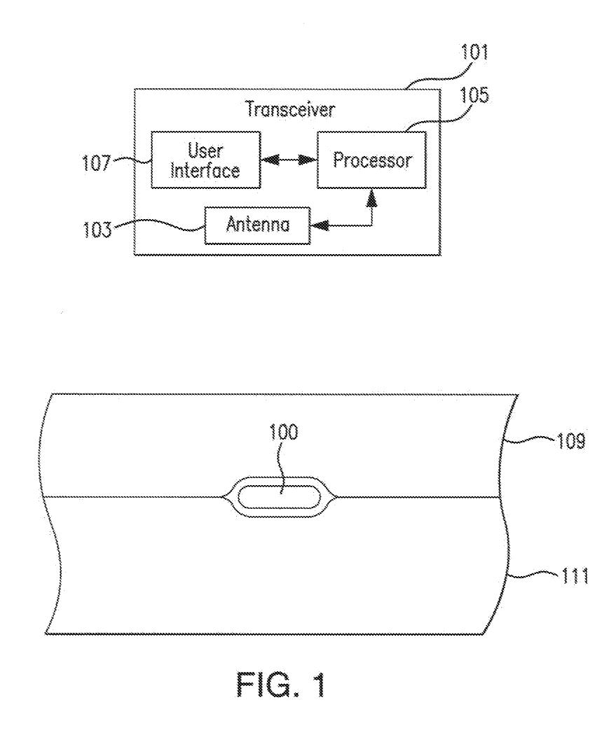

FIG. 1 is a schematic view of a sensor system embodying aspects of the present invention. In some embodiments, the system may include a sensor 100 and an external transceiver 101. In some embodiments, as shown in FIG. 1, the sensor 100 may be configured for implantation in a living animal (e.g., a living human). The sensor 100 may be implanted, for example and without limitation, in a living animal's arm, wrist, leg, abdomen, or other region of the living animal suitable for sensor implantation. For example, as shown in FIG. 1, in some non-limiting embodiments, the sensor 100 may be implanted between the skin 109 and subcutaneous tissues 111. In some embodiments, the sensor 100 may be a fully implantable sensor. However, this is not required, and, in some alternative embodiments, the sensor 100 may be a partially implantable (e.g., transcutaneous) sensor. In some embodiments, the sensor 100 may take measurements indicative of an amount or concentration of an analyte (e.g., glucose) in a medium (e.g., interstitial fluid) of the living animal. In some embodiments, the sensor 100 may be an electro-optical sensor. In some alternative embodiments, the sensor 100 may be an electrochemical.

In some embodiments, the transceiver 101 may be an externally worn transceiver (e.g., attached via an armband, wristband, waistband, or adhesive patch). In some embodiments, the transceiver 101 may remotely power and/or communicate with the sensor to initiate and receive one or more measurements (e.g., analyte measurements and/or temperature measurements) from the sensor (e.g., via near field communication (NFC)). However, this is not required, and, in some alternative embodiments, the transceiver 101 may power and/or communicate with the sensing system 105 via one or more wired connections. In some non-limiting embodiments, the transceiver 101 may be a smartphone (e.g., an NFC-enabled smartphone). In some embodiments, the transceiver 101 may communicate information (e.g., one or more analyte measurements) wirelessly (e.g., via a Bluetooth™ communication standard such as, for example and without limitation Bluetooth Low Energy) to a hand held application running on a display device (e.g., smartphone).

In some embodiments, the transceiver 101 may include one or more of an antenna 103, a processor 105, and a user interface 107. In some non-limiting embodiments, the user interface 107 may include a liquid crystal display (LCD), but, in other embodiments, different types of displays may be used.

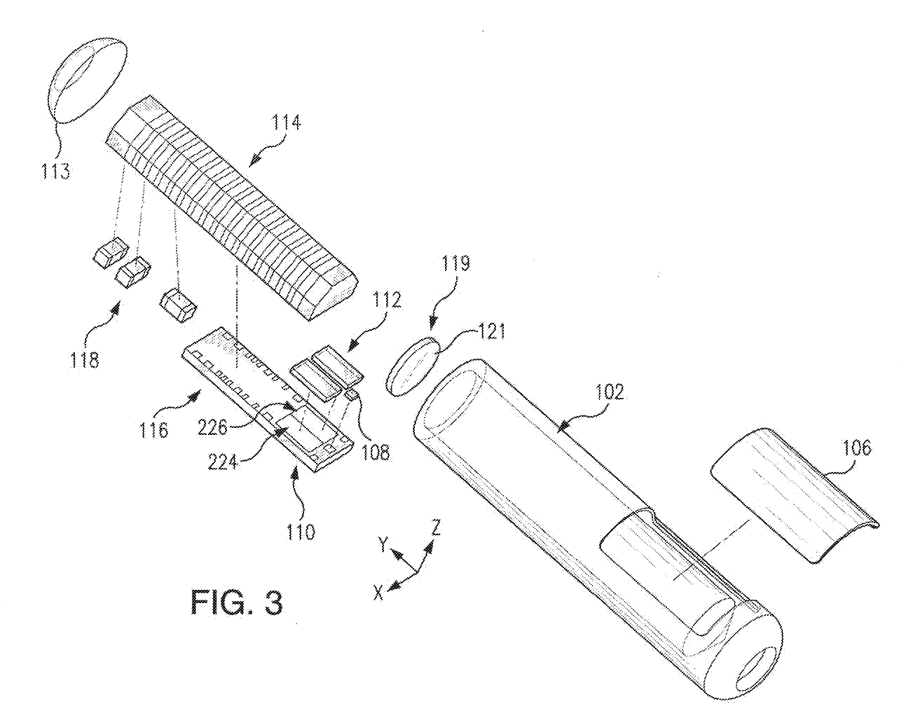

In some embodiments, the antenna 103 may include an inductive element, such as, for example, a coil. The antenna 103 may generate an electromagnetic wave or electrodynamic field (e.g., by using a coil) to induce a current in an inductive element (e.g., inductive element 114 of FIGS. 3-8) of the sensor 100, which may power the sensor 100. The antenna 103 may also convey data (e.g., commands) to the sensor 100. For example, in some non-limiting embodiments, the antenna 103 may convey data by modulating the electromagnetic wave used to power the sensor 100 (e.g., by modulating the current flowing through a coil of the antenna 103). The modulation in the electromagnetic wave generated by the transceiver 101 may be detected/extracted by the sensor 100. Moreover, the antenna 103 may receive data (e.g., measurement information) from the sensor 100. For example, in some non-limiting embodiments, the antenna 103 may receive data by detecting modulations in the electromagnetic wave generated by the sensor 100, e.g., by detecting modulations in the current flowing through the coil of the antenna 103. In some embodiments, the inductive element of the antenna 103 and the inductive element (e.g., inductive element 114 of FIGS. 3-8) of the sensor 100 may be in any configuration that permits adequate field strength to be achieved when the two inductive elements are brought within adequate physical proximity.

In some embodiments, the processor 105 may calculate one or more analyte concentrations based on the analyte sensor data received from the sensor 100. In some embodiments, the processor 105 may also generate one or more alerts and/or alarms based on the calculated analyte concentrations (e.g., if the calculated analyte concentration exceeds or falls below one or more thresholds). The calculated analyte concentrations, alerts, and/or alarms may be displayed via the user interface 107 and/or conveyed to a remote display device (e.g., a mobile device such as, for example and without limitation, a smartphone).

In some embodiments, the transceiver 101 may communicate (e.g., using a wireless communication standard, such as, for example, Bluetooth) with a remote device (e.g., a smartphone, personal data assistant, handheld device, or laptop computer). The remote device may receive calculated analyte concentrations, alerts, and/or alarms from the transceiver 101 and display them. Display by the remote device may be in addition to, or in the alternative to, display by the user interface 107 of the transceiver 101. For example, in some embodiments, as illustrated in FIG. 1, the transceiver 101 may include a user interface 107, but this is not required. In some alternative embodiments, the transceiver 101 may not have a user interface 107, and calculated analyte concentrations, alerts, and/or alarms may instead be displayed by a remote device.

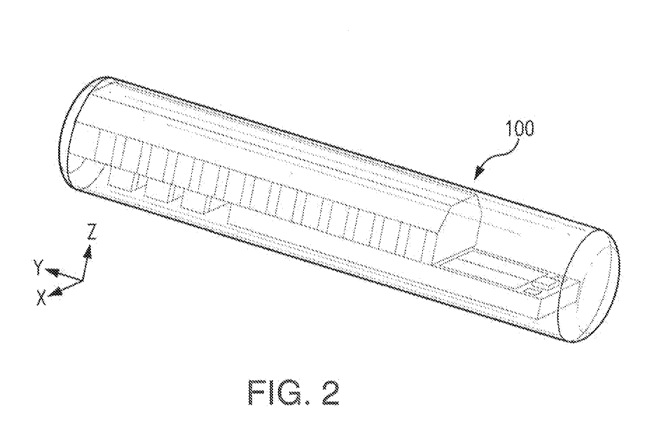

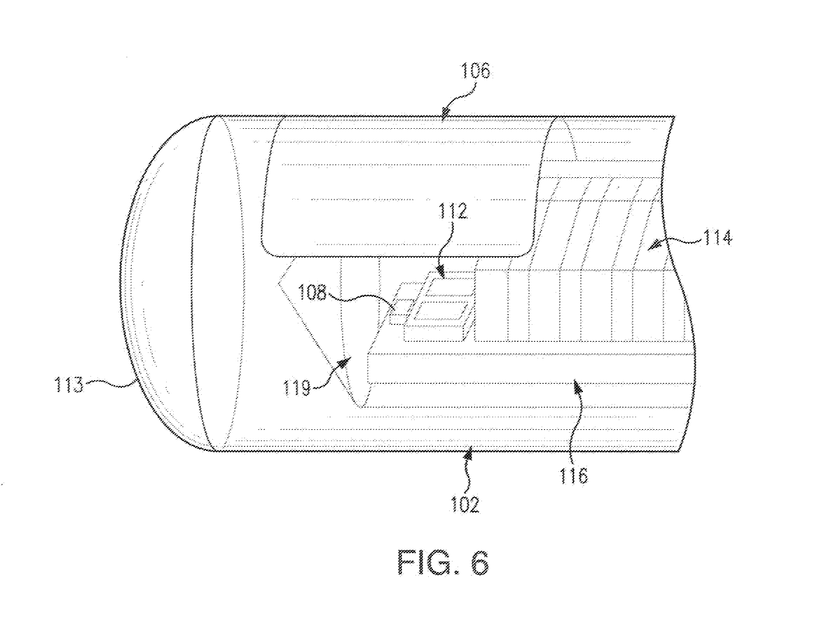

FIGS. 2-7 illustrate a non-limiting embodiment of a sensor 100 embodying aspects of the present invention that may be used in the sensor system illustrated in FIG. 1. In some embodiments, the sensor 100 may be an optical sensor. In one non-limiting embodiment, sensor 100 includes a sensor housing 102 (i.e., body, shell, or capsule). In exemplary embodiments, sensor housing 102 may be formed from a suitable, optically transmissive polymeric material, such as, for example, acrylic polymers (e.g., polymethylmethacrylate (PMMA)). In some embodiments, as shown in FIGS. 3 and 6, the sensor housing 102 may include a cap or cover 113 at an end thereof.

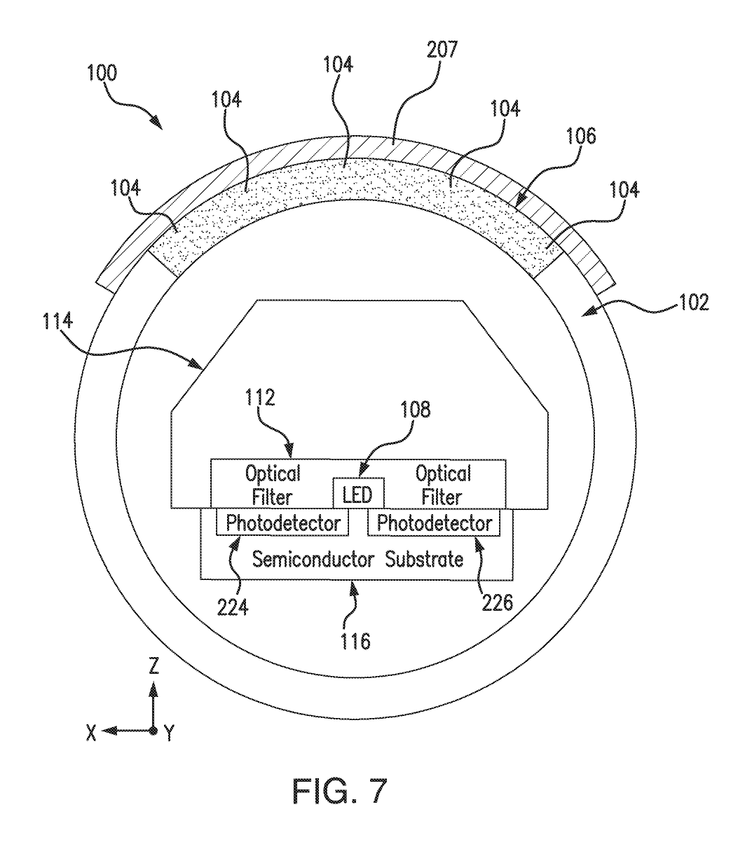

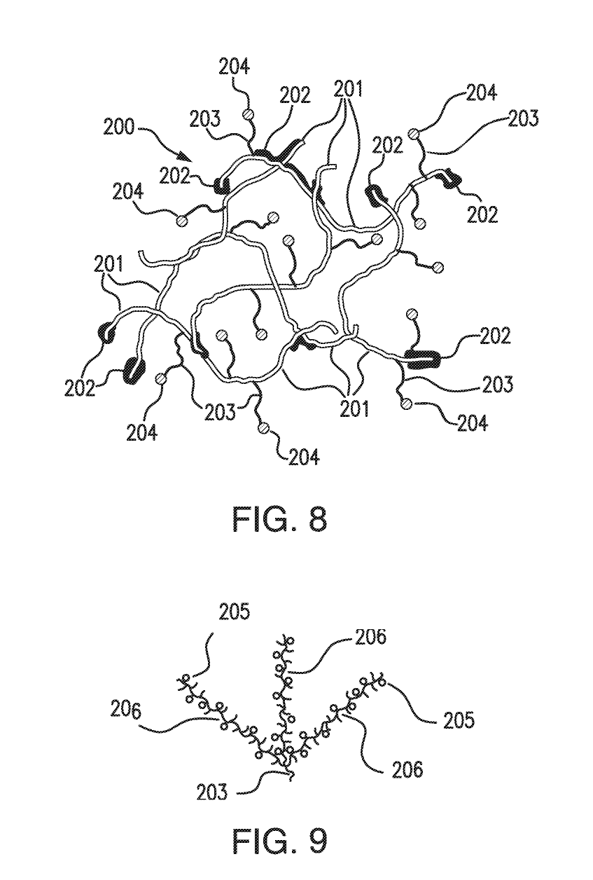

In some embodiments, as illustrated in FIGS. 2-7, the sensor 100 may include indicator molecules 104 (see, e.g., FIGS. 7 and 8). Indicator molecules 104 may be fluorescent indicator molecules or absorption indicator molecules. In some non-limiting embodiments, the indicator molecules 104 may be as described in U.S. Pat. No. 6,344,360 or U.S. patent application Ser. No. 13/937,871, which are incorporated herein by reference in their entireties. In some non-limiting embodiments, sensor 100 may include an analyte indicator 106. In some non-limiting embodiments, the analyte indicator 106 may be a polymer graft (e.g., a matrix layer or hydrogel) or a porous base membrane layer coated or embedded on at least a portion of the exterior surface of the sensor housing 102, with the indicator molecules 104 distributed throughout the graft. The analyte indicator 106 may be embedded within the sensor housing 102 and/or cover the entire surface of sensor housing 102 or only one or more portions of the surface of housing 102. Similarly, the indicator molecules 104 may be distributed throughout the entire analyte indicator 106 or only throughout one or more portions of the analyte indicator 106.

In some embodiments, as illustrated in FIGS. 3-7, the sensor 100 may include a light source 108, which may be, for example, a light emitting diode (LED) or other light source that emits light over a range of wavelengths that interact with the indicator molecules 104.

In some embodiments, as illustrated in FIGS. 3-7, the sensor 100 may include one or more photodetectors 110 (e.g., photodiodes, phototransistors, photoresistors, or other photosensitive elements) which, in the case of a fluorescence-based sensor, is sensitive to fluorescent light emitted by the indicator molecules 104 such that a signal is generated by the photodetector 110 in response thereto that is indicative of the level of fluorescence of the indicator molecules and, thus, the amount of analyte of interest (e.g., glucose).

In some embodiments, as illustrated in FIGS. 3, 6, and 7, the sensor 100 may include one or more optical filters 112, such as high pass or band pass filters, that may cover a photosensitive side of the one or more photodetectors 110.

In some embodiments, as shown in FIG. 2, the sensor 100 may be wholly self-contained. In other words, the sensor may be constructed in such a way that no electrical leads extend into or out of the sensor housing 102 to supply power to the sensor (e.g., for driving the light source 108) or to convey signals from the sensor 100. Instead, in some embodiments, the sensor 100 may be powered by an external power source (e.g., external transceiver 101). For example, the external power source may generate a magnetic field to induce a current in an inductive element 114 (e.g., a coil or other inductive element). Additionally, the sensor 100 may use the inductive element 114 to communicate information to an external sensor reader (e.g., transceiver 101). In some embodiments, the external power source and data reader may be the same device (e.g., transceiver 101).

In some embodiments, sensor 100 may include a semiconductor substrate 116 and circuitry may be fabricated in the semiconductor substrate 116. The circuitry may include analog and/or digital circuitry. Also, although in some preferred embodiments the circuitry is fabricated in the semiconductor substrate 116, in alternative embodiments, a portion or all of the circuitry may be mounted or otherwise attached to the semiconductor substrate 116. In other words, in alternative embodiments, a portion or all of the circuitry may include discrete circuit elements, an integrated circuit (e.g., an application specific integrated circuit (ASIC)) and/or other electronic components discrete and may be secured to the semiconductor substrate 116, which may provide communication paths between the various secured components.

In some embodiments, the one or more photodetectors 110 may be mounted on the semiconductor substrate 116, but, in some preferred embodiments, the one or more photodetectors 110 may be fabricated in the semiconductor substrate 116. In some embodiments, the light source 108 may be mounted on the semiconductor substrate 116. For example, in a non-limiting embodiment, the light source 108 may be flip-chip mounted on the semiconductor substrate 116. However, in some embodiments, the light source 108 may be fabricated in the semiconductor substrate 116.

In some embodiments, the sensor 100 may include one or more capacitors 118. The one or more capacitors 118 may be, for example, one or more tuning capacitors and/or one or more regulation capacitors. Further, the one or more capacitors 118 may be in addition to one or more capacitors fabricated in the semiconductor substrate 116.

In some embodiments, the sensor 100 may include a reflector 119 (i.e., mirror). Reflector 119 may be attached to the semiconductor substrate 116 at an end thereof (see, FIG. 3). In a non-limiting embodiment, reflector 119 may be attached to the semiconductor substrate 116 so that a face portion 121 of reflector 119 is generally perpendicular to a top side of the semiconductor substrate 116 (i.e., the side of semiconductor substrate 116 on or in which the light source 108 and one or more photodetectors 110 are mounted or fabricated) and faces the light source 108. The face 121 of the reflector 119 may reflect radiation emitted by light source 108. In other words, the reflector 119 may block radiation emitted by light source 108 from reaching the axial end of the sensor 100.

According to one aspect of the invention, an application for which the sensor 100 was developed (although by no means the only application for which it is suitable) is measuring various biological analytes in the living body of an animal (including a human). For example, sensor 100 may be used to measure glucose, oxygen toxins, pharmaceuticals or other drugs, hormones, and other metabolic analytes in, for example, the human body. The specific composition of the analyte indicator 106 and the indicator molecules 104 therein may vary depending on the particular analyte the sensor is to be used to detect and/or where the sensor is to be used to detect the analyte (i.e., in interstitial fluid). Preferably, however, analyte indicator 106 should facilitate exposure of the indicator molecules to the analyte. Also, it is preferred that the optical characteristics of the indicator molecules (e.g., the level of fluorescence of fluorescent indicator molecules) be a function of the concentration of the specific analyte to which the indicator molecules are exposed.

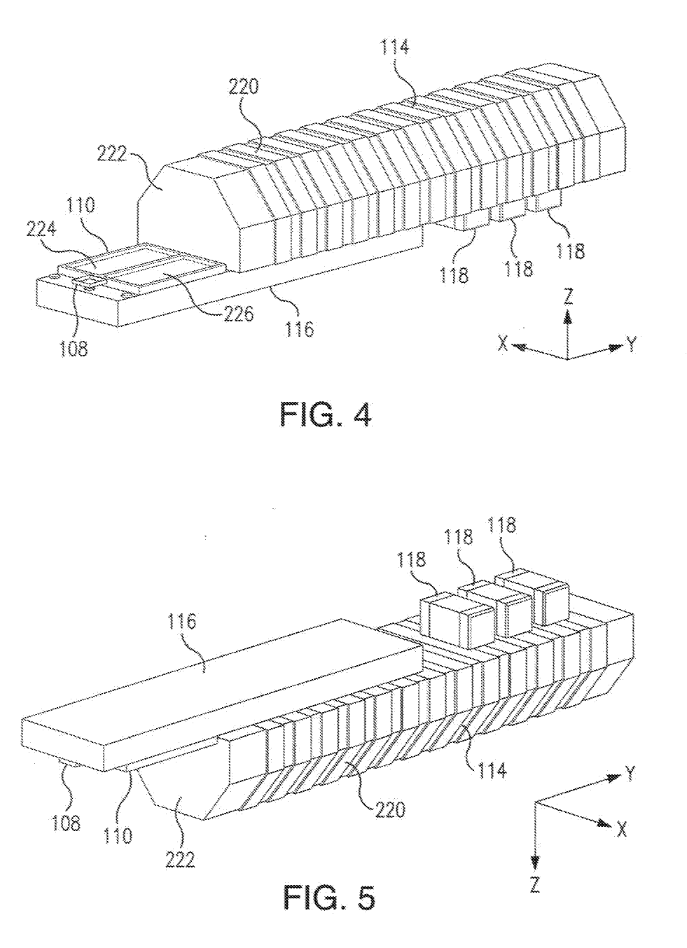

FIGS. 4 and 5 illustrate perspective views of the sensor 100 according to some non-limiting embodiments. In FIGS. 4 and 5, the sensor housing 102, filters 112, and the reflector 119, which may be included in some embodiments of the sensor 100, are not illustrated. In some embodiments, as shown in FIGS. 4 and 5, the inductive element 114 may comprise a coil 220. In some embodiments, the coil 220 may be a copper coil, but, in some alternative embodiments, other conductive materials, such as, for example and without limitation, screen printed gold, may be used. In some embodiments, the coil 220 is formed around a ferrite core 222. Although core 222 is ferrite in some embodiments, in some alternative embodiments, other core materials may be used. In some embodiments, coil 220 is not formed around a core. Although coil 220 is illustrated as a cylindrical coil in FIGS. 4 and 5, in other embodiments, coil 220 may be a different type of coil, such as, for example, a flat coil.

In some embodiments, coil 220 is formed on ferrite core 222 by printing the coil 220 around the ferrite core 222 such that the major axis of the coil 220 (magnetically) is parallel to the longitudinal axis of the ferrite core 222. A non-limiting example of a coil printed on a ferrite core is described in U.S. Pat. No. 7,800,078, which is incorporated herein by reference in its entirety. In an alternative embodiment, coil 220 may be a wire-wound coil. However, embodiments in which coil 220 is a printed coil as opposed to a wire-wound coil are preferred because each wire-wound coil is slightly different in characteristics due to manufacturing tolerances, and it may be necessary to individually tune each sensor that uses a wire-wound coil to properly match the frequency of operation with the associated antenna. Printed coils, by contrast, may be manufactured using automated techniques that provide a high degree of reproducibility and homogeneity in physical characteristics, as well as reliability, which may be important for implant applications, and may increase cost-effectiveness in manufacturing.

In some embodiments, a dielectric layer may be printed on top of the coil 220. The dielectric layer may be, in a non-limiting embodiment, a glass based insulator that is screen printed and fired onto the coil 220. In an exemplary embodiment, the one or more capacitors 118 and the semiconductor substrate 116 may be mounted through the dielectric.

In the illustrated embodiment, the one or more photodetectors 110 include a first photodetector 224 and a second photodetector 226. First and second photodetectors 224 and 226 may be mounted on or fabricated in the semiconductor substrate 116.

FIGS. 6 and 7 illustrate side and cross-sectional views, respectively, of the sensor 100 according to one embodiment. As illustrated in FIGS. 6 and 7, the light source 108 may be positioned to emit light that travels within the sensor housing 102 and reaches the indicator molecules 104 of the analyte indicator 106, and the first and second photodetectors 224 and 226, which may be located beneath filters 112, may be positioned to receive light from the indicator molecules 104 of the analyte indicator 106.

In operation, the light source 108 (e.g., an LED) may emit excitation light that travels within the sensor housing 102 and reaches the indicator molecules 104 of the analyte indicator 106. In a non-limiting embodiment, the excitation light may cause the indicator molecules 104 distributed in analyte indicator 106 to fluoresce. As the analyte indicator 106 may be permeable to the analyte (e.g., glucose) in the medium (e.g., blood or interstitial fluid) into which the sensor 100 is implanted, the indicator molecules 104 in the analyte indicator 106 may interact with the analyte in the medium and, when irradiated by the excitation light, may emit indicator fluorescent light indicative of the presence and/or concentration of the analyte in the medium.

The photodetectors 224 and 226 are used to receive light (see FIG. 3). Each photodetector 224 and 226 may be covered by a filter 112 that allows only a certain subset of wavelengths of light to pass through (see FIG. 3). The filters 112 may be thin film (e.g., dichroic) filters deposited on glass, and the filters 112 may pass only a narrow band of wavelengths and otherwise reflect the received light. The filters 112 may be identical (e.g., both filters 112 may allow signal light to pass) or different (e.g., one filter 112 may allow signal light to pass, and the other filter 112 may allow reference light to pass).

In some embodiments, the photodetector 226 may be a reference photodetector, and the filter 112 may pass light at the same wavelength as the wavelength of the excitation light 329 emitted from the light source 108 (e.g., 378 nm). In some embodiments, the photodetector 224 may be a signal photodetector that detects the amount of fluoresced light 331 that is emitted from the indicator molecules 104 in the analyte indicator 106. In some non-limiting embodiments, the signal filter 112 (i.e., the filter 112 covering photodetector 224) may pass light in the range of about 400 nm to 500 nm. Higher analyte levels may correspond to a greater amount of fluorescence of the molecules 104 in the analyte indicator 106, and therefore, a greater amount of photons striking the signal photodetector 224.

In some non-limiting embodiments, as illustrated in FIG. 7, the sensor 100 may include a coating 207 on the outside of the analyte indicator 106. In some non-limiting embodiments, the coating 207 may be on all or a portion of the outside of the analyte indicator 106. In some non-limiting embodiments where a portion of the sensor housing 102 is not covered by the analyte indicator 106, the coating 207 may additionally be on all or a portion of the portion of the sensor housing 102 not covered by the analyte indicator 106. In some embodiments, the coating 207 may include a catalytically active material configured to reduce deterioration of the analyte indicator 106 by catalyzing degradation of reactive oxygen species (ROS). In some embodiments, the catalytically active material in the coating may include, for example and without limitation, one or more of platinum, iridium, palladium, manganese oxide, thiol and/or disulfide containing compounds, and catalase. In some non-limiting embodiments, the coating 207 may be a sputter coating sputtered on the outside of the analyte indicator 106.

Embodiments of the present invention may include one or more of several possible solutions to analyte indicator deterioration, as explained above, white blood cells, including neutrophils, may attack an implanted sensor 100. The neutrophils release, inter alia, hydrogen peroxide, which may degrade indicator molecules (e.g., by oxidizing a boronate group of an indicator molecule and disabling the ability of the indicator molecule to bind glucose).

FIG. 8 illustrates an analyte indicator 200 in accordance with embodiments of the present invention. In some embodiments, the analyte indicator 200 may be used as the analyte indicator 106 of the sensor 100 illustrated in FIGS. 1-7. In some non-limiting embodiments, the analyte indicator 200 may be embedded within and/or covering at least a portion of the housing 102 for a sensor 100. In some embodiments, the sensor 100 may include the coating 207 on the outside of the analyte indicator 200. In some non-limiting embodiments, the analyte indicator 200 may include one or more of a porous base 201, catalytically active material 202, a polymer unit 203, and an analyte sensing element 204.

In some non-limiting embodiments, the porous base 201 may comprise fibril nylon (e.g., Nylon 6,6) having an exterior surface and an interior surface. However, this is not required, and, in some alternative embodiments, the porous base 201 may comprise other, similar membrane materials, such as, for example and without limitation, cellulose acetate, polypropylene, polyether sulfone, polyethylene, polyvinylidene difluoride (PVDF), polycarbonate, polytetrafluoroethylene (PTFE), or polyethylene terephthalate (PET). In some non-limiting embodiments, the porous base 201 does not vary in opacity. In some non-limiting embodiments, the porous base 201 may retain its physical, chemical, and optical properties in the presence of compression. As illustrated in FIG. 8, in some embodiments, the porous base 201 may include long, connected strands.

In some embodiments, the analyte indicator 200 may include a catalytically active material 202 disposed on at least one of the exterior surface and interior surface of the porous base 201. The catalytically active material 202 may be configured to catalyze the degradation of ROS, thereby protecting against indicator molecule degradation. In some non-limiting embodiments, the catalytically active material 202 may comprise platinum. However, this is not required, and, in some alternative embodiments, the catalytically active material 202 may comprise one or more of iridium, palladium, silver, manganese oxide, thiol and/or disulfide containing compounds and copolymers, catalase, and any other physiologically compatible metal or metal oxide that is capable of catalyzing the decomposition of ROS. In some embodiments, the catalytically active material 202 may be incorporated on the porous base 201 as a coating. The catalytically active material 202 may be applied to the porous base 201 in any suitable fashion, such as, for example and without limitation, by sputter deposition. In some non-limiting embodiments, the thickness of the catalytically active material 202 may be within a range, for example and without limitation, from 0.5 nm to 15 nm, and this range should be understood as describing and disclosing all range values (including all decimal or fractional values) and sub-ranges within this range.

In some embodiments, the analyte indicator 200 may include a catalytically active material 202 coated on the exterior surface of the porous base 201 (e.g., a thin layer, such as a 10 nm thick layer of platinum). In some embodiments, the analyte indicator 200 may additionally or alternatively include a catalytically active material 202 coated interior surface of the porous base 201 (e.g., a thin layer, such as a 3 nm thick layer of platinum). In some embodiments, the analyte indicator 200 may include a catalytically active material 202 coated on both the exterior surface (e.g., a thin layer, such as a 10 nm thick layer of platinum) and the interior surface (e.g., a thin layer, such as a 3 nm thick layer of platinum) of the porous base 201.

In some embodiments, the analyte indicator 200 may additionally or alternatively include a scavenging material disposed on at least one of the interior and exterior surfaces of the porous base 201. In some embodiments, the scavenging material may be configured to consume ROS. In some embodiments, the scavenging material may include one or more of the following: boronic acid containing compounds, di-acid containing compounds, tocopherol and its derivatives, and ascorbic acid and its derivatives.

In some embodiments, as illustrated in FIG. 8, the analyte indicator 200 may include a polymer 203 attached or polymerized onto or out of the porous base 201. In some embodiments, the polymer 203 may be in units (e.g., strands) that are attached to or polymerized off of the backbone provided by the porous base 201. In some non-limiting embodiments, the polymer 203 may be polyethylene glycol (PEG). However, this is not required, and, in alternative embodiments, other materials may be used, such as, for example and without limitation, poly(oxazolines), poly(acrylamides), poly(electrolytes), poly(ethers), poly(vinyl pyrolidone), Poly(ethylenimines), poly(vinyl alcohol), poly(acrylates and methacrylates), and/or poly(maleic anhydride). The polymer units 203 may provide a flexible structure that retains its physical, chemical, and/or optical properties when compressed. In some embodiments, the polymer units may be hydrophilic or amphiphilic. In FIG. 8, the polymer units 203 are shown as short strands off of the long strands of the porous base 201.

In some embodiments, the analyte indicator 200 may include one or more analyte sensing elements 204. The one or more analyte sensing elements 204 may be attached or copolymerized to the polymer units 203. In some non-limiting embodiments, as illustrated in FIG. 8, each polymer unit 203 may have one analyte sensing element 204 attached or copolymerized thereto. However, this is not required, and, in some alternative embodiments, one or more of the polymer units 203 may not have an analyte sensing element 204 attached or copolymerized thereto. For example, in one non-limiting alternative embodiment, a small number of analyte sensing elements 204 may be attached to the polymer units 203 (e.g., an analyte sensing element 204 may be attached to approximately one tenth of the polymer units 203). Moreover, in some alternative embodiments, one or more of the polymer units 203 may have multiple (e.g., two, three, four or more) analyte sensing elements 204 attached or copolymerized thereto. In FIG. 8, the analyte sensing elements 204 are shown as circles attached or copolymerized to the polymer units 203.

In some embodiments, one or more of the analyte sensing elements 204 may consist of one or more indicator molecules 205 attached to a polymer unit 203. In some embodiments, the indicator molecules 205 are comprised of a fluorescent lanthanide metal chelate complex. However, this is not required, in other embodiments, the indicator molecules may be a relatively hydrophilic molecule or structure that reversibly binds to glucose and in response, becomes fluorescent such that the indicator molecule emits light in a range of 400 nm to 500 nm. In some embodiments, as illustrated in FIG. 9, one or more of the analyte sensing elements 204 may include one or more indicator polymer chains (i.e., linear chains) 206 attached or polymerized onto or out of a polymer unit 203. In this way, the indicator polymer chains 206 may branch out from the polymer units 203. Accordingly, in some embodiments, the analyte indicator 200 may have a branched polymer structure. The indicator polymer chains 206 may include one or more indicator molecules 205 attached thereto. Although FIG. 9 illustrates an analyte sensing element 204 having three indicator polymer chains 206 attached or polymerized onto or out of a polymer unit 203, this is not required, and, in some alternative embodiments, an analyte sensing element 204 may have a different number (e.g., one, two, four, five, etc.) of indicator polymer chains 206 attached or polymerized onto or out of a polymer unit 203. In some embodiments, although not illustrated in FIG. 9, one or more of indicator polymer chains 206 may have one or more indicator polymer chains 206 attached or polymerized onto or out of the indicator polymer chain 206 for additional branching.

In some non-limiting embodiments, the indicator polymer chains 206 may be short (e.g., 1-200 nm). In some embodiments, the overall structure of the analyte indicator 200 including the one or more indicator polymer chains 206 retains its physical, chemical, and/or optical properties in the presence of compression from an external source (e.g., a secondary membrane wrapped on top of the analyte indicator). In some embodiments, the polymer chains 206 could consist of, for example and without limitation, 2-hydroxyethylmethacrylate, poly(ethylene glycol) methacrylate, acrylic acid, methacrylic acid, [2-(methacrylolyloxy)ethyl]dimethyl-(3-sulfopropyl)ammonium hydroxide, or vinyl pyrrolidone. However, in some alternative embodiments, other materials may be used for the polymer chains. In some embodiments, the indicator polymer chains 206 may be hydrophilic or amphiphilic.

In some non-limiting embodiments, the analyte indicator 200 may be formed by making the polymer chain(s) 206 with the indicator molecules 205 attached thereto and then attaching polymer chain(s) 206 to the polymer unit(s) 203, which may be already be attached or polymerized onto or out of the porous base 201. However, this is not required, and, in alternative embodiments, the analyte indicator 200 may be formed in different manners.

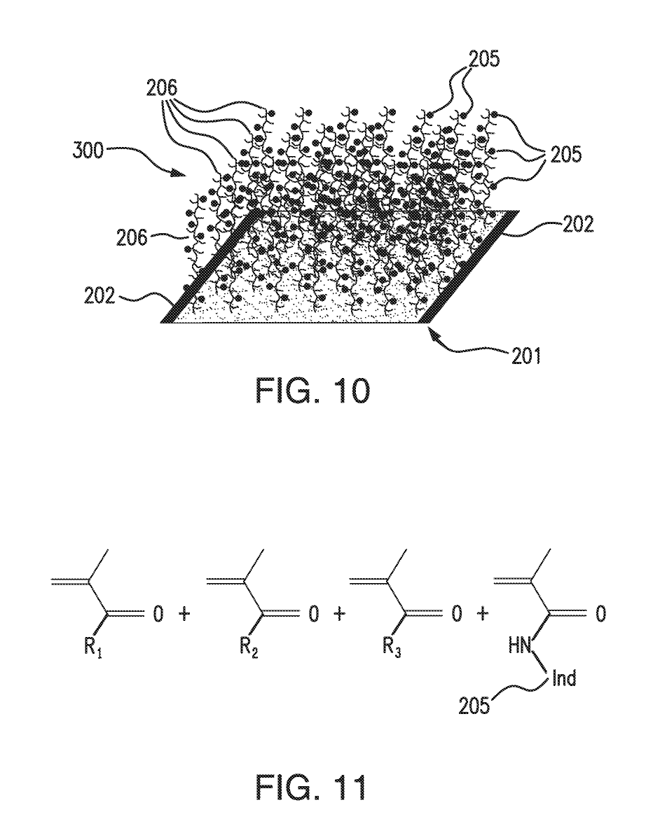

FIG. 10 illustrates an analyte indicator 300 embodying aspects of the present invention. In some embodiments, the analyte indicator 300 may be used as the analyte indicator 106 of the sensor 100 illustrated in FIGS. 1-7. In some embodiments, the sensor 100 may include the coating 207 on the outside of the analyte indicator 300. Similar to the analyte indicator 200 illustrated in FIG. 8, in some embodiments, the analyte indicator 300 may include a porous base 201. The analyte indicator 300 may include one or more indicator polymer chains or strands 206 attached or polymerized onto or out of the porous base 201. Accordingly, the analyte indicator 300 may have a linear polymer structure. In some embodiments, as shown in FIG. 10, the catalytic active material 202 may be disposed one or more of the exterior and interior surfaces of the porous base 201 of the analyte indicator 300. In some embodiments, as shown in FIG. 10, the catalytically active material 202 may coat the porous base 201 with the indicator molecule polymer chains or strands 206 sticking out.

In some embodiments, the analyte indicator 300 may additionally or alternatively include a scavenging material disposed on at least one of the interior and exterior surfaces of the porous base 201. In some embodiments, the scavenging material may be configured to consume ROS. In some embodiments, the scavenging material may include one or more of the following: boronic acid containing compounds, di-acid containing compounds, tocopherol and its derivatives, and ascorbic acid and its derivatives.

In some embodiments, as illustrated in FIG. 11, the linear polymer chains 206 may be grafted onto a surface of the porous base 201. FIG. 11 shows the base monomer make-up of a grafted linear copolymer 206. In some embodiments, R₁, R₂, and R₃ may be hydrophilic acrylate-based monomers such as but not limited to 2-hydroxyethyl methacrylate (HEMA), poly(ethylene glycol) methacrylate (PEGMA), and/or acrylic/methacrylic acid. In some non-limiting embodiments, the indicator polymer chains 206 of the analyte indicator 200 illustrated in FIG. 9 may have the base monomer make-up illustrated in FIG. 11.

In some non-limiting embodiments, the analyte indicator 300 may be formed by making the polymer chain(s) 206 with the indicator molecules 205 attached thereto and then attaching polymer chain(s) 206 to the porous base 201. However, this is not required, and, in alternative embodiments, the analyte indicator 300 may be formed in a different manner.

In some embodiments, the analyte indicator may be attached to the analyte sensor by O₂ plasma treating the sensor followed by tack welding the analyte indicator to the sensor at 450° F. (230° C.). However, this is not required, and, in alternative embodiments, the analyte indicator may be attached to the analyte sensor using a different method. In some embodiments, analyte indicator is attached to the sensor in a manner that allows intimate contact of the analyte indicator (e.g., analyte indicator 200, which may have the branched polymer structure, or analyte indicator 300, which may be a linear copolymer graft membrane) with the encasement (e.g., the PMMA encasement) of the sensor platform (e.g., by cutting the analyte indicator to 0.18″×0.47″ when used with a sensor undercut width of 0.193″).

In some embodiments, the analyte indicator (e.g., analyte indicator 200, which may have the branched polymer structure, or analyte indicator 300, which may be a linear copolymer graft membrane) has one or more of the following advantages: (i) ability to be produced on a large scale and stored, (ii) elimination of hydration before implant (i.e., allows for dry implant), (iii) retention of its physical, chemical, and optical properties in the presence of compression, (iv) optical stability, (v) built-in oxidative stability, (vi) fast response times, and (vii) a tuneable K_{d}.

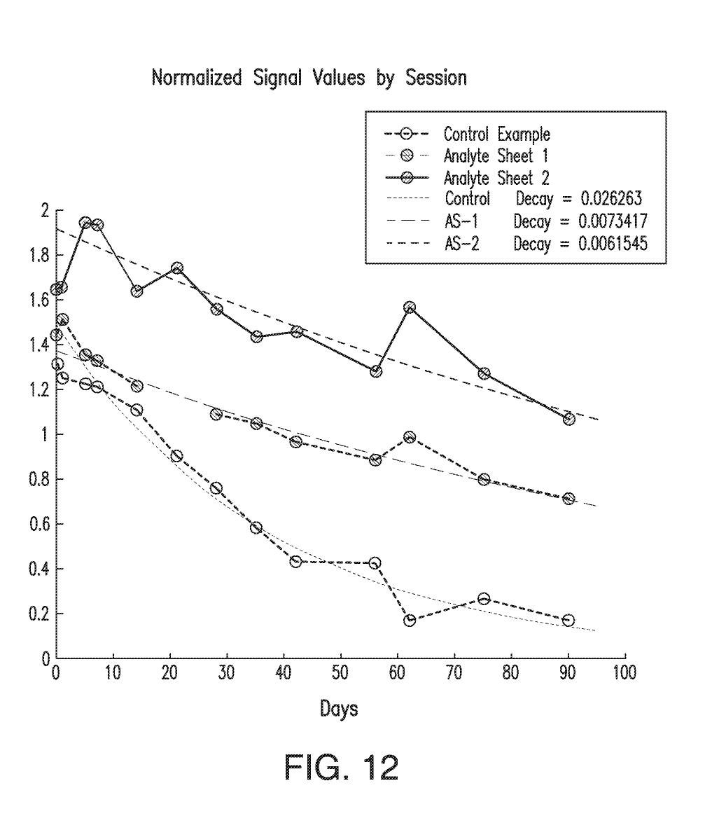

FIG. 12 illustrates a non-limiting example of the results of in vitro experimental testing to evaluate the protection of an implanted sensor from ROS degradation in humans by the use of a catalytically active material 202 incorporated into an analyte indicator 106 (e.g. analyte indicator 200 of FIG. 8 or analyte indicator 300 of FIG. 10). Three analyte indicator configurations were tested: (1) a control analyte indicator hydrogel having a 10 nm coating of platinum sputtered on the outside of the control analyte indicator but no catalytically active material incorporated into the control analyte indicator hydrogel, (2) a first analyte indicator (“Analyte Sheet 1” or “AS-1”) having a 10 nm coating of platinum sputtered on the outside of the first analyte indicator and a 10 nm layer of platinum disposed on the exterior surface of the porous base 201 of the first analyte indicator, and (3) a second analyte indicator (“Analyte Sheet 2” or “AS-2”) having a 10 nm coating of platinum sputtered on the outside of the second analyte indicator, a 10 nm layer of platinum disposed on the exterior surface of the porous base 201 of the second analyte indicator, and a 3 nm layer of platinum on the interior surface of the porous base 201 of the second analyte indicator.

The three analyte indicator configurations were tested in an in vitro environment, in which the analyte indicators were each submerged in a solution containing an oxidizing agent (e.g., a hydrogen peroxide buffer) to simulate exposure to ROS in a human body. The signal intensities of the three analyte indicator configurations were measured with a fluorimeter in the presence of glucose in the solution over four day period, and the in vitro results are shown in Table 1 below.

In particular, Table 1 below shows the oxidative half-lives of the Control, Analyte Sheet 1, and Analyte Sheet 2. As shown in Table 1, the Control and Analyte Sheet 1 have similar half-lives. The half-life of Analyte Sheet 2, which included the 10 nm coating of platinum and the 10 nm and 3 nm layers of platinum on the exterior and interior surfaces of the porous base, is more than twice the half-lives of the Control and Analyte Sheet 1.

TABLE 1

| Configuration | Avg. half-life | Avg. % mod. remaining |
|---|---|---|
| Control Example | 8.1 hrs | 0% |
| Analyte Sheet 1 | 8.1 hrs | 1.5% (±0.03) |
| Analyte Sheet 2 | 17.0 hrs | 8.3% (±5.8) |

The three analyte indicator configurations were also tested in an in vivo environment in which the three analyte indicator configurations (i.e., the control analyte indicator hydrogel with the 10 nm coating of platinum sputtered on the outside, Analyte Sheet 1, and Analyte Sheet 2) were implanted into guinea pigs for a duration of ninety-four days with sensor reads at specific time points to assess in vivo signal degradation. FIG. 12 shows the normalized signal values generated by the three analyte indicator configurations over the ninety-four day period. The normalized signal values for the Control, Analyte Sheet 1, and Analyte Sheet 2 are shown in blue, red, and yellow, respectively. Both Analyte Sheets 1 and 2 generated stronger normalized signal values over the ninety-four day period than the Control. As shown in FIG. 12, the generated signal values from the Control deteriorated significantly faster over the ninety-four day period than the signal values generated by Analyte Sheets 1 and 2. Relative to the normalized signals from Analyte Sheet 1, the normalized signals from Analyte Sheet 2 were higher over the ninety-four day period.

These studies demonstrate the effectiveness in combining a fibril nylon porous base with a catalytically active material, such as platinum, to preserve the operability of the analyte sensor that is exposed to a ROS. As shown in Table 1, increasing the amount of the catalytically active material (e.g., platinum) may prolong the longevity of the analyte indicator. The increased amount of catalytically active material may catalyze more decomposition of hydrogen peroxide, thereby preventing and/or reducing oxidation of the indicator molecules in the analyte indicator.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

What is claimed is:

1. An analyte indicator comprising:
a porous base having an interior surface and an exterior surface;
a catalytically active material disposed on at least one of the interior and exterior surfaces of the porous base, wherein the catalytically active material is configured to catalyze the degradation of reactive oxygen species (ROS);
a polymer unit polymerized onto or out of the porous base; and
an analyte sensing element attached to the polymer unit and/or copolymerized with the polymer unit.

2. The analyte indicator of claim 1, wherein the porous base comprises one or more of nylon, cellulose, cellulose acetate, polypropylene, polyethylene, poly(ethylene terephthalate), poly(ether sulfone), poly(vinylidene difluoride), and poly(tetrafluoroethylene).

3. The analyte indicator of claim 1, wherein the polymer unit is a polyethylene glycol (PEG) unit.

4. The analyte indicator of claim 1, wherein the catalytically active material comprises one or more of platinum, iridium, palladium, manganese oxide, thiol and/or disulfide containing compounds, and catalase.

5. The analyte indicator of claim 1, further comprising a scavenging material disposed on at least one of the interior and exterior surfaces of the porous base, wherein the scavenging material is configured to consume ROS.

6. The analyte indicator of claim 5, wherein the scavenging material comprises one or more of the following: boronic acid containing compounds, di-acid containing compounds, tocopherol and its derivatives, and ascorbic acid and its derivatives.

7. The analyte indicator of claim 1, wherein the analyte sensing element comprises one or more indicator molecules configured to reversibly bind to an analyte and exhibit one or more detectable properties indicative of whether analyte is bound.

8. The analyte indicator of claim 1, wherein the porous base is flexible.

9. The analyte indicator of claim 1, wherein the analyte indicator retains its physical, chemical, and optical properties in the presence of compression.

10. The analyte indicator of claim 1, wherein the analyte sensing element includes one or more indicator polymer chains, and the one or more indicator polymer chains include one or more indicator molecules configured to reversibly bind to an analyte and exhibit one or more detectable properties indicative of whether analyte is bound.

11. The analyte indicator of claim 1, wherein further comprising a coating of catalytically active material on the analyte indicator.

12. The analyte indicator of claim 11, wherein the coating of catalytically active material on the analyte indicator is sputtered onto the analyte indicator.

13. A sensor comprising:
a sensor housing; and
an analyte indicator embedded within and/or covering at least a portion of the sensor housing, wherein the analyte indicator comprises:
a porous base having an interior surface and an exterior surface;
a catalytically active material disposed on at least one of the interior and exterior surfaces of the porous base, wherein the catalytically active material is configured to catalyze the degradation of reactive oxygen species (ROS);
a polymer unit polymerized onto or out of the porous base; and
an analyte sensing element attached to the polymer unit or copolymerized with the polymer unit.

14. The sensor of claim 13, further comprising a light source configured to emit excitation light to the analyte indicator; and a photodetector configured to receive fluorescent light emitted by the analyte indicator.

15. The sensor of claim 13, further comprising a coating of catalytically active material on the analyte indicator.

16. The sensor of claim 15, wherein the coating of catalytically active material on the analyte indicator is sputtered onto the analyte indicator.

17. The sensor of claim 13, further comprising a scavenging material disposed on at least one of the interior and exterior surfaces of the porous base, wherein the scavenging material is configured to consume ROS.

18. The sensor of claim 17, wherein the scavenging material comprises one or more of the following: boronic acid containing compounds, di-acid containing compounds, tocopherol and its derivatives, and ascorbic acid and its derivatives.

19. An analyte indicator comprising:
a porous base;
an indicator polymer chain attached or polymerized onto or out of the porous base;
one or more indicator molecules attached to the indicator polymer chain;
a catalytically active material disposed on at least one of the interior and exterior surfaces of the porous base, wherein the catalytically active material is configured to catalyze the degradation of reactive oxygen species (ROS).

20. The analyte indicator of claim 19, wherein the indicator polymer chain is a first indicator polymer chain, and the analyte indicator further comprises:
a second indicator polymer chain attached or polymerized onto or out of the porous base; and
indicator molecules attached to the second indicator polymer chain.

21. The analyte indicator of claim 19, wherein the porous base comprises one or more of nylon, cellulose, cellulose acetate, polypropylene, polyethylene, poly(ethylene terephthalate), poly(ether sulfone), poly(vinylidene difluoride), and poly(tetrafluoroethylene).

22. The analyte indicator of claim 19, wherein the catalytically active material comprises one or more of platinum, iridium, palladium, manganese oxide, thiol and/or disulfide containing compounds, and catalase.

23. The analyte indicator of claim 19, wherein the one or more indicator molecules are configured to reversibly bind to an analyte and exhibit one or more detectable properties indicative of whether analyte is bound.

24. The analyte indicator of claim 19, wherein the porous base is flexible.

25. The analyte indicator of claim 19, wherein the analyte indicator retains its physical, chemical, and optical properties in the presence of compression.

26. The analyte indicator of claim 19, wherein further comprising a coating of catalytically active material on the analyte indicator.

27. The analyte indicator of claim 26, wherein the coating of catalytically active material on the analyte indicator is sputtered onto the analyte indicator.

28. The analyte indicator of claim 19, further comprising a scavenging material disposed on at least one of the interior and exterior surfaces of the porous base, wherein the scavenging material is configured to consume ROS.

29. The analyte indicator of claim 28, wherein the scavenging material comprises one or more of the following: boronic acid containing compounds, di-acid containing compounds, tocopherol and its derivatives, and ascorbic acid and its derivatives.

30. A sensor comprising:
a sensor housing; and
an analyte indicator embedded within and/or covering at least a portion of the sensor housing, wherein the analyte indicator comprises:
a porous base;
an indicator polymer chain attached or polymerized onto or out of the porous base;
one or more indicator molecules attached to the indicator polymer chain;
a catalytically active material disposed on at least one of the interior and exterior surfaces of the porous base, wherein the catalytically active material is configured to catalyze the degradation of reactive oxygen species (ROS).

31. The sensor of claim 30, further comprising a light source configured to emit excitation light to the analyte indicator; and a photodetector configured to receive fluorescent light emitted by the analyte indicator.

32. The sensor of claim 30, further comprising a coating of catalytically active material on the analyte indicator.

33. The sensor of claim 32, wherein the coating of catalytically active material on the analyte indicator is sputtered onto the analyte indicator.

34. The sensor of claim 30, further comprising a scavenging material disposed on at least one of the interior and exterior surfaces of the porous base, wherein the scavenging material is configured to consume ROS.

35. The sensor of claim 34, wherein the scavenging material comprises one or more of the following: boronic acid containing compounds, di-acid containing compounds, tocopherol and its derivatives, and ascorbic acid and its derivatives.

36. A sensor comprising:
a sensor housing;
an analyte indicator embedded within and/or covering at least a portion of the sensor housing, wherein the analyte indicator comprises:
a porous base,
one or more indicator molecules configured to exhibit one or more detectable properties based on an amount or concentration of an analyte in proximity to the indicator molecules, and
a catalytically active material disposed on at least one of the interior and exterior surfaces of the porous base, wherein the catalytically active material is configured to catalyze the degradation of reactive oxygen species (ROS);
a coating of catalytically active material on an exterior surface of the analyte indicator, wherein the catalytically active material is configured to catalyze the degradation of ROS.

37. The sensor of claim 36, further comprising a scavenging material disposed on at least one of the interior and exterior surfaces of the porous base, wherein the scavenging material is configured to consume ROS.

38. The sensor of claim 37, wherein the scavenging material comprises one or more of the following: boronic acid containing compounds, di-acid containing compounds, tocopherol and its derivatives, and ascorbic acid and its derivatives.
